(19) United States
(12) Patent Application Publication
(10) Pub. No.: US 2021/0187414 A1
(43) Pub. Date: Jun. 24, 2021

(54) SYSTEMS AND METHODS FOR EXTRACTION OF COMPOUNDS FROM BOTANICAL MATTER

(71) Applicant: RADD Scientific Inc., Langley (CA)

(72) Inventors: David BRUCE, Langley (CA); Toby ASTILL, Victoria (CA); Martin RASKA, Langley (CA); Dean SCODELLARO, Surrey (CA)

(21) Appl. No.: 17/267,674

(22) PCT Filed: Aug. 28, 2019
(86) PCT No.: PCT/CA2019/051187

§ 371 Date: Feb. 10, 2021

Related U.S. Application Data
(60) Provisional application No. 62/724,936, filed on Aug. 30, 2018.

(51) Int. Cl. B01D 11/02; C07D 311/78; C07C 37/00; G01N 21/3577

(52) U.S. Cl. CPC B01D 11/0207; B01D 11/0288; C07D 311/78; G01N 2021/3595; G01N 21/3577; B01D 11/0203; C07C 37/004

(57) ABSTRACT

Systems and methods of extracting compounds from botanical matter are provided. The system includes: a solvent source; an extraction vessel which receives botanical matter from which compounds, including at least one target compound are extracted into a solution; a detector that obtains real time information the compounds; a separation vessel for separating the target compound from the solution; a throttle located between the extraction vessel and the separation vessel for controlling flow of the solution; and a controller connected to the detector and the at the throttle for receiving and processing the real time information and controlling, via the throttle, flow rate of the solution from the extraction vessel to the separation vessel based at least partly on the processed real time information about the compounds.

### TECHNICAL FIELD

This invention relates to systems and methods for extraction of compounds from botanical matter, such as cannabis.

### BACKGROUND

Variability in botanical matter raises challenges for efficiently extracting desired compounds. For example, continuing to run an extraction after a desired compound has been fully extracted wastes energy and time. Extracting undesired compounds necessitates additional separation processes to remove them. Improved systems and methods for efficient extraction of compounds from botanical matter are desirable.

### BRIEF DESCRIPTION OF THE DRAWINGS

The accompanying drawings illustrate non-limiting example embodiments of the invention.

FIG. 1 is a block diagram of an extraction system according to an embodiment of the invention.

FIG. 2 is a state diagram showing the major operating modes of an extraction system according to an embodiment of the invention.

FIG. 3 is a block diagram of an isolated section of an extraction vessel according to an embodiment of the invention.

FIG. 4 is a plot of FTIR data from both in-situ and ex-situ measurements according to an embodiment of the invention.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

Some aspects of the invention relate to extraction systems operable at high throughput efficiency and reliability in a cost-effective manner. The systems are configured to increase extraction efficiency by adjusting reaction parameters such as reaction time with real time information about the extraction as it proceeds. Real time information about the extraction is obtained by in situ sensors, and based on this information the extraction is controlled in a manner that, for example, allows the run to be stopped when one or more desirable compounds are fully extracted or when one or more undesirable compounds are being extracted or reach undesirable levels.

As used herein, the term “cannabis” means a part (e.g. leaf, stem, root, flower) of and/or any product from a Cannabis species (e.g., Cannabis sativa L., Cannabis indica Lam., Cannabis ruderalis Janish.), and includes both “marijuana” and “hemp”, as well as any variety, cultivar and hybrid of such species.

As used herein, the term “real time” means a level of processing responsiveness sufficiently immediate for a particular process or determination (e.g. a detector obtaining signals relating to an extracted compound and communicating those signals to a controller).

The concentration of desirable or target compounds present in botanical matter can vary due to biological factors such as botanical matter species and strain, and environmental factors such as growing conditions (e.g. nutrients, lighting, watering) and timing of harvest. In cannabis extraction, for example, certain cannabinoids (e.g. tetrahydrocannabinol and/or cannabidiol), terpenes and flavonoids may be considered target compounds and the concentration of these compounds can vary between different sources and batches of cannabis. As such, in order to fully extract the target compounds, process parameters such as extraction time, temperature and pressure, can vary.

In addition to the variation in the concentration of target compounds present, variation can exist in the nature and concentration of undesirable compounds that may be extracted. In cannabis extraction, for example, certain alkaloids and monoterpenes may be considered undesirable compounds. The concentration of undesirable compounds present in the botanical matter is also variable due to biological factors and environmental factors. As such, there is variation in the length of operation time permissible before extraction of undesirable compounds begins to occur, or occurs to an undesirable threshold level. Other variable process parameters such as temperature and pressure, may also affect the degree of extraction of undesirable compounds.

Signal detection and measurement of extracted compounds using a probe can also be influenced by a variety of process conditions, including: probe occlusion by fouling by extracted compounds or particulates from the botanical matter; complex flow-based movement of the compounds; state conditions of the extraction system, namely variations in temperature and pressure, influenced for example by temperature and density of the botanical matter; and variation in the physical placement of the botanical matter in relation to the probe.

Regarding the complexity of flow-based movement of extracted compounds, for example, filling of the extraction vessel with solvent, such as supercritical, gaseous, or liquid carbon dioxide, causes fluidic momentum in the extraction vessel. This fluidic momentum can be represented by an in-vessel flow. In-vessel flow conditions adjacent to the probe, or the in-vessel flow conditions between the botanical matter and the probe, has variation from extraction to extraction due to the disorganized nature of the packing of botanical matter according to batch-to-batch filling process conditions. Filling of botanical matter could also be operated in a continuous filling manner with similar variations due to the disorganized packing of the botanical matter.

Signal measurement, if used to determine concentration of an extracted target compound alone, would be unable to predict the time required for complete extraction of the target compound due to lack of knowledge of absolute concentrations of the target compound and variability in the measurements as discussed above. Batch to batch variability in water content, particle size, and biological structure (e.g. roots, shoots, etc.) can further exacerbate these challenges.

The order in which the compounds are extracted from botanical matter is determined by the properties of the compounds themselves and is invariant.

The diffusion of extracted compounds in solution is determined by the molecular mass and polarity of the molecule. The diffusion of the extracted compounds towards and away from a probe occurs according to Fick's Laws and is invariant.

Aspects of the invention relate to signal measurement of a plurality of discrete compounds to provide a matrix of information relating to the extracted compounds. The inventors have determined that ratios of the measurements of extracted compounds, and changes over time thereof, can provide useful information regarding the rate at which target compounds are being extracted, and that this information in turn can be used to derive adjustments to extraction process parameters such as adjustments to pressure, temperature and extraction time to increase extraction efficiency.

In some embodiments, monitoring the ratio of measurements (e.g. concentrations) of two marker compounds being extracted, at a particular time point or over time depending on the embodiment, can give information regarding a target compound, or target compound for which a signal has been lost.

In some embodiments, monitoring one or more ratios of measurements of two or more marker compounds being extracted, at a particular time point or over time depending on the embodiment, can be used to derive the time that will be taken for complete extraction of a target compound which has yet to be fully extracted from the botanical matter.

In some embodiments, monitoring the ratio of measurements of a marker compound and a target compound, at a particular time point or over time depending on the embodiment, can be used to derive the time that will be taken for complete extraction of the target compound which has yet to be fully extracted from the botanical matter.

In some embodiments, monitoring one or more ratios of two or more marker compounds, at a particular time point or over time depending on the embodiment, can be used to determine when full extraction of a target compound will be complete and/or when an undesirable compound begins to be extracted or begins to approach undesirable concentrations.

Thus precise predictions of extraction times, i.e., cycle endpoints, can be derived without the need for precision in absolute measurements because reliance is on ratios and/or changes, rather than absolute values, of output signals. Stopping extraction once full extraction of the target compound(s) is complete allows for savings in energy and time in processing. Stopping extraction before undesirable compounds are extracted or reach undesirable concentrations avoids the need for additional separation processes to remove the undesirable compounds from solution.

In some embodiments, monitoring one or more ratios of two or more extracted compounds, at a particular time point or over time depending on the embodiment, can be used to assess the efficiency of process conditions, and based on this information adjustments to pressure and/or temperature of the extraction vessel may be made. For example, adjustments to temperature and/or pressure may be made to increase rate of extraction of more volatile target compounds. Or, for example, detection of certain components, or certain components in certain ratios, or rates of change of certain ratios of certain components, may be a signal to adjust temperature and/or pressure. For example, detection of non-decarboxylated species can be a trigger for increasing temperature and/or pressure to activate or increase rate of decarboxylation.

In some embodiments, programming of computer algorithms used to examine the ratios of measured extracted compounds used as markers for determination of full extraction of target compounds can be facilitated by development of databases of results of prior testing of similar botanical matter. In example embodiments, tetrahydrocannabinol (THC) may be the last cannabinoid to be extracted, so if other cannabinoids are required preferentially, the THC signal will be the marker compound for full extraction of the more mobile cannabinoids. In some embodiments, development of such databases may be assisted by computational machine learning. Algorithm development facilitated by the use of machine learning allows for rapid automation optimization of extraction processes, independent of botanical strain or local processing conditions or known relative extraction ratios of known compounds.

In some embodiments, the concentration (and thus the measured signal) of the extracted compound in the extraction vessel is too low to be accurately measured and so isolation of a small portion of the extraction vessel and alteration of the environmental conditions therein to enhance the signal can be performed. For example, adjustments in the local pressure and temperature of the isolated section of the extraction vessel can cause phase separation of extracted compounds, increasing the strength of the measured signal. The term “phase separation” as used herein includes processes such as condensation, precipitation, sublimation, distillation and the like. The resulting separated material includes materials such as condensate, precipitate, sublimate, distillate and the like.

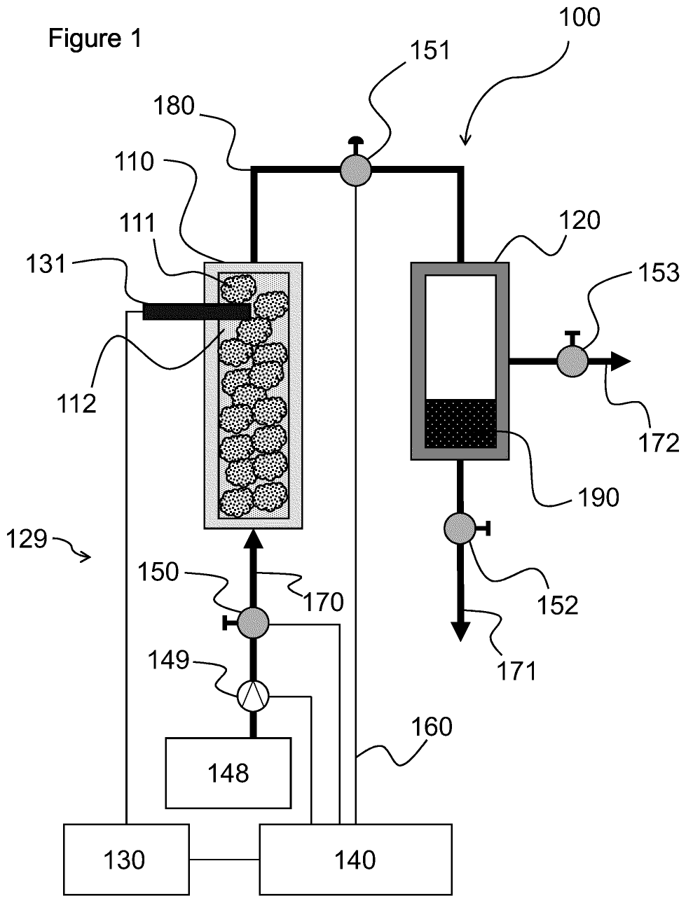

FIG. 1 is a block diagram of an extraction system according to one embodiment of the invention. The system 100 includes an extraction vessel 110 in fluid communication with a separation vessel 120.

A solvent source 148 is in fluid communication with extraction vessel 110 via a closed conduit 170. A valve 150 regulates flow of solvent 112 from solvent source 148 to extraction vessel 110. A pump 149 may be provided to deliver a pressurized flow of solvent 112 to extraction vessel 110. Pressure of solvent 112 may range for example from 1 atm to 700 atm, or from 74 atm to 340 atm. Solvent 112 may for example be fluidic carbon dioxide.

Extraction vessel 110 is configured to receive solvent 112 and botanical matter 111. Botanical matter 111 may for example be cannabis. In some embodiments, solvent 112 may be a mix of solvents. In particular embodiments, the solvent mix may include hydrocarbons, such as alcohols, in combination with carbon dioxide. The cannabis may be mechanically processed cannabis with a size distribution in the range of 10 to 5000 microns. Once compounds begin to be extracted from botanical matter 111 and dissolve in solvent 112, solvent 112 is referred to herein as solution 112′. Extracted compounds in the case of cannabis as botanical matter may include cannabinoids (including tetrahydrocannabinol and/or cannabidiol), terpenes and flavonoids. The concentration of extracted compounds in solution 112′ may vary in operation of the system from 0.01% w/w to 50% w/w or more.

In other embodiments, alternative solvents, alternative botanical matter, and/or alternative compounds may be extracted in the invention.

Extraction vessel 110 may be a pressure vessel of a fixed volume. In some embodiments extraction vessel 110 may be a steel capped container or a plurality of steel capped containers connected in parallel or series.

A detector 129 is associated with extraction vessel 110. Detector 129 includes a probe 131 and a measurement unit 130. In some embodiments, detector 129 may be provided may be FTIR, LC, GC, MS, UV absorbance, UV fluorescence, IR-spectral analysis, or any other combination thereof. Probe 131 is inserted into an interior of extraction vessel 110. The location of probe 131 within extraction vessel 110 needs to be in an area where the flow of solvent 112/solution 112′ passes by, and preferably not a dead zone in extraction vessel 110 such as adjacent to the inlet for solvent 112. In some embodiments probe 131 is placed for example from 1 nm to 50 cm, or from 100 nm to 100 um, away from botanical matter 111.

In some embodiments probe 131 may be positioned in the interior of extraction vessel 110 (as illustrated in FIG. 1). In some embodiments probe 131 may be positioned in closed conduit 180 anywhere upstream of throttle 151. In some embodiments there may be one or more additional throttle elements (not shown) positioned in closed conduit 180 between extraction vessel 110 and throttle 151 downstream of extraction vessel 110 and upstream of throttle 151. Any section of the closed conduit 180 downstream of extraction vessel 110 but upstream of throttle 151 forms part of the extraction vessel volume and as such probe 131 may be integrated into closed conduit 180 without divergence from the invention.

Detector 129 is in communication (e.g. wired or wireless) with a controller 140, and controller 140 is in turn in communication (e.g. wired by cable 160 or wireless) with a throttle 151 provided on a closed conduit 180 that connects extraction vessel 110 to separation vessel 120. Controller 140 includes a processor (not shown). Throttle 151 may for example be a valve. Based on analysis of results from monitoring by measurement unit 130 of detector 129, as discussed above, controller 140 mediates actuation of throttle 151 (as well as any additional throttle elements in closed conduit 180 as discussed above) to control flow of solution 112′ from extraction vessel 110 to separation vessel 120. In some embodiments, controller 140 may additionally or alternatively be in communication with valve 150 and/or pump 149 to control pressure in extraction vessel 110, and closed circuits 170 and 180. In some embodiments, controller 140 may additionally or alternatively be in communication with a heater and/or cooler (not shown) to control temperature in extraction vessel 110, and closed circuits 170 and 180.

Separation vessel 120 may have a fixed volume, and in some embodiments may be a steel capped container, or a plurality of steel capped containers connected in parallel or series. Solution 112′ laden with extracted compounds is phase separated in separation vessel 120, for example due to decrease in pressure. Separation vessel 120 may for example maintain a pressure in the range of 1 atm to 70 atm, or 20 atm to 60 atm. Separation vessel 120 has two outlets: one leading to a closed conduit 172 with a valve 153 for discharging solvent 112; and another leading to closed conduit 171 with a valve 152 for recovering separated extracted compounds 190 separated from solvent 112.

In some embodiments one extraction vessel is connected via closed conduit to the separation means. It will be apparent to those skilled in the art that the arrangement of extraction vessels and separation vessels could contain one or multiple extraction vessels in series or parallel connection with one or multiple separation vessel interconnected by closed conduit without divergence from the invention.

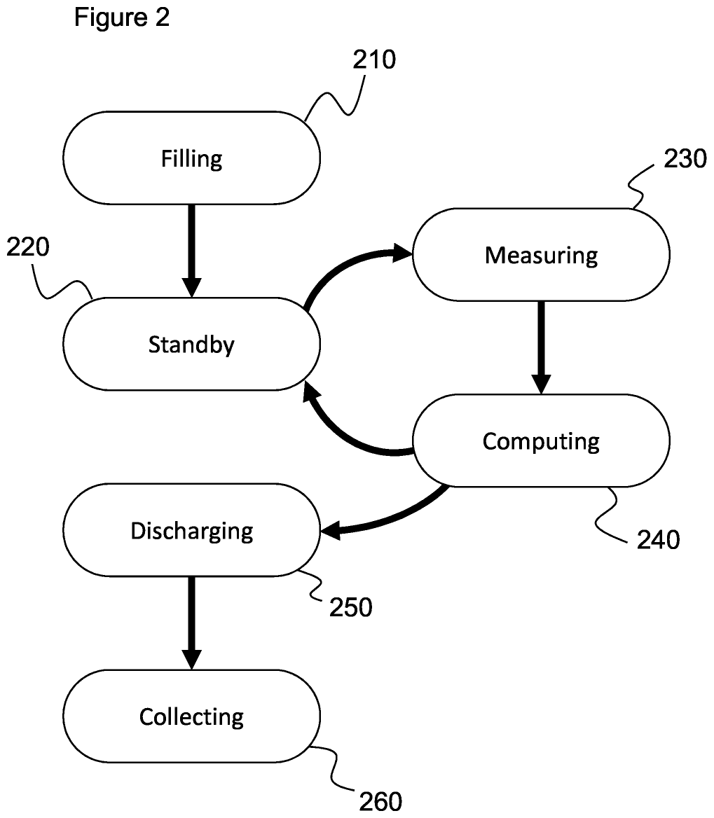

FIG. 2 is a state diagram describing the major operating modes of systems, and thus a method, according to one embodiment of the invention. The following description will refer to system 100 for convenience but can refer to systems according to any embodiment of the invention.

The six states of Filling 210, Standby 220, Measuring 230, Computing 240, Discharging 250, and Collecting 260 represent the normal or ‘successful’ flow of events.

The Filling state 210 is the system state where extraction vessel 110 is being filled or emptied with botanical matter 111. The Filling state is the state in which extraction vessel 110 will become pressurized with solvent 112 from solvent source 148 by opening valve 150, after botanical matter 111 is received within and extraction vessel 110 is sealed. Throttle 151 is closed during Standby state 220.

The Standby state 220 is the state in which extraction vessel 110 is filled with botanical matter 111 and solvent 112, and chemical absorption is occurring and solvent 112 becomes a solution 112′ comprising compounds extracted from botanical matter 111. Standby state 220 is the default state for the system and begins once the pressure in extraction vessel 110 reaches a predetermined system operating value. Throttle 151 remains closed during Standby state 220. The duration of Standby state 220 may for example range from 1 minute to 1440 minutes, or 5 minutes to 60 minutes. The duration will depend on factors including the size of botanical matter 111, the volume of extraction vessel 110, and the targeted components.

The Measuring state 230 is the state in which detector 129 is actively taking measurements of extracted compounds. The measurements from detector 129 are sent to controller 140 in real time. Throttle 151 may remain open or closed during Measuring state 230.

The Computing state 240 is the state in which the signals from Measuring state 230 are analyzed by the processor of controller 140 and determination of the next processing step occurs.

If the algorithmic determination of set points concludes the process requires further extraction, the operation reverts to the Standby state 220.

If the algorithmic determination of set points determines the extraction is complete, the operation proceeds to the Discharging state 250. Throttle 151 may remain open or closed during Computing state 240.

The Discharging state 250 is the state in which throttle 151 and any other additional throttle elements of closed conduit 180 controlling flow between extraction vessel 110 and separation vessel 120 are opened to allow for solution 112′ (laden with extracted compounds) to flow into separation vessel 120. The Discharging state includes phase separation of the extracted compounds 190 from solution 112′ (due to the pressure drop from extraction vessel 110 to separation vessel 120). Solution 112′ thus reverts to solvent 112 and is discharged through conduit 172 by operation of valve 153. In some cases, while the system is being discharged, throttle 150 may be controlled to maintain constant system pressure in extraction vessel 110.

The Collecting state 260 is the state in which system 100 is substantially discharged, and extracted compounds 190 may be recovered from (for example a bottom ⅓ of) separation vessel 120 through conduit 171 by operation of valve 152.

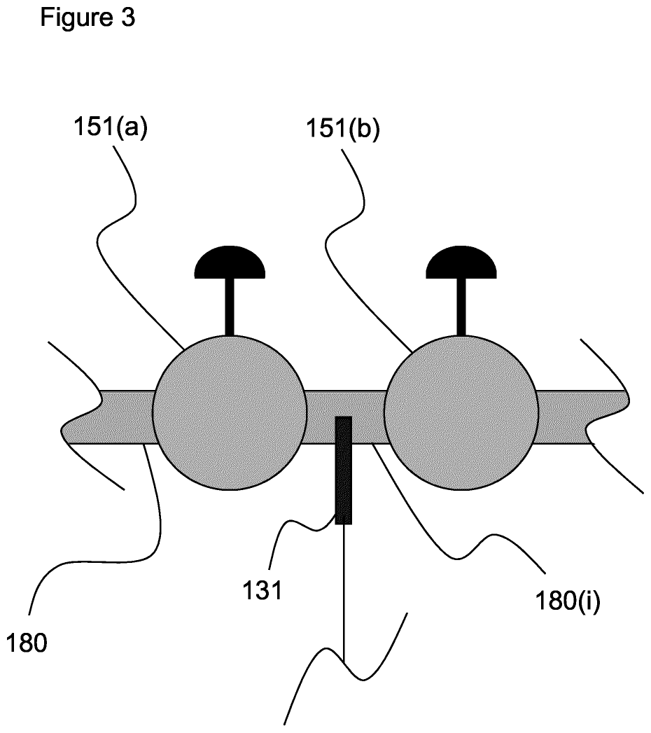

FIG. 3 is a block diagram of an isolated section 180(i) of an extraction system according to one embodiment of the invention. In some embodiments probe 131 may be positioned in closed conduit 180 anywhere upstream of throttle 151(b). In some embodiments there may be one or more additional throttle elements such as throttle 151(a) positioned in closed conduit 180 between extraction vessel 110 (not shown) and throttle 151(b). An isolated section of the extraction vessel 180(i) is then formed in which the temperature and/or pressure of isolated section 180(i) can be changed independently of the environmental conditions of the rest of closed conduit 180 and extraction vessel 110 (not shown). In some embodiments, the temperature and/or pressure of the isolated section is reduced in order to phase separate extracted compounds near and/or on the probe to facilitate detection of a more intense probe signal. Isolated section of extraction vessel 180(i) may be caused to have a reduced pressure for example in the range of 1 atm to 72 atm, or 20 atm to 60 atm, and/or a reduced temperature in the range of 31° C. to −56° C., or 31° C. to 0° C.

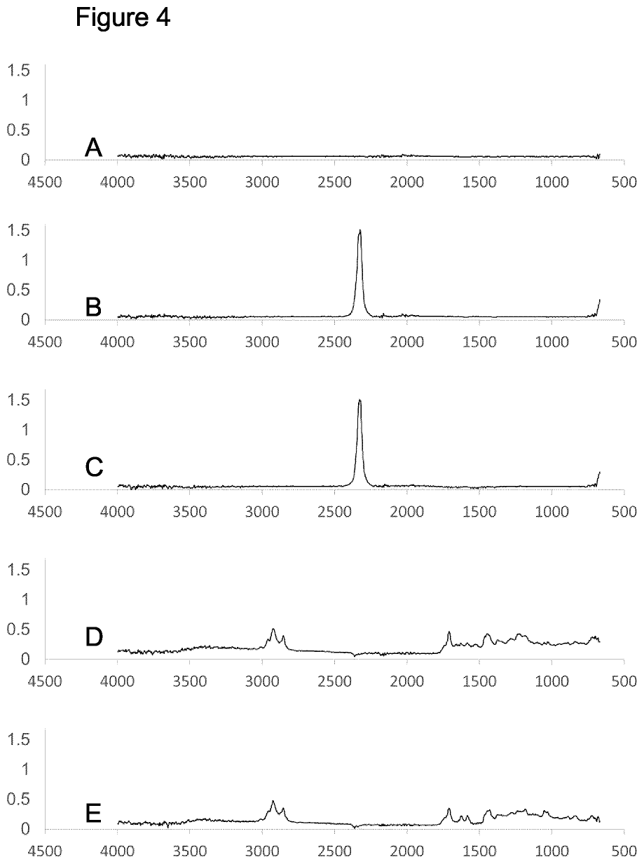

FIG. 4 shows an example of results of in-situ FTIR probe measurements of system operation in comparison with an ex-situ measurement of extracted compounds 190. In each plot the y-axis represents measured absorption (A.U.) and the x-axis represents wave number (cm⁻¹). Plot A shows the measurement when the system is initially loaded with botanical matter 111. Plot B shows the measurement when the system is initially pressurized with solvent 112, in this case fluidic CO₂. Plot C shows measurement of the CO₂ pressurized system after 8 hours when solvent 112 has been allowed to absorb extracted compounds 190, to become solution 112′. Plots D and E are essentially at the same time point as Plot C, but Plot D shows in-situ measurement of compounds 190 phase separated from solution 112′ through reduction in system pressure, and Plot E is the corresponding ex-situ measurement of the extracted compounds 190 recovered from separator vessel 120. The large peak at around 2300 cm⁻¹ in Plots B and C indicate the presence of supercritical CO₂ and the disappearance of this peak in Plots D and E is consistent with pressure reduction causing supercritical CO₂ to become non-detected gaseous CO₂. Importantly, Plot D, compared to Plot C, shows a distinct enhancement in in-situ measured signal of extracted compounds 190 (e.g. the peaks at around 2800 cm⁻¹ to around 3000 cm⁻¹ and at around 1700 cm⁻¹ and below), and these more intense probe signals are congruent with the corresponding ex-situ measured signals in Plot E.

Where a component is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

This application is intended to cover any variations, uses, or adaptations of the invention using its general principles. Further, this application is intended to cover such departures from the present disclosure as come within known or customary practice in the art to which this invention pertains and which fall within the limits of the appended claims. Accordingly, the scope of the claims should not be limited by the preferred embodiments set forth in the description, but should be given the broadest interpretation consistent with the description as a whole.

1. An extraction system comprising:
a solvent source;
an extraction vessel in fluid communication with the solvent source, the extraction vessel configured to receive botanical matter from which a solvent from the solvent source extracts a plurality of compounds into a solution, the plurality of compounds including at least one target compound;
a detector configured to obtain real time information about at least one of the plurality of compounds in the solution;
a separation vessel in fluid communication with the extraction vessel, the separation vessel configured to separate the at least one target compound from the solution;
at least one throttle located between the extraction vessel and the separation vessel for controlling flow of the solution from the extraction vessel to the separation vessel; and
a controller in operative communication with the detector and the at least one throttle, the controller configured to receive and process the real time information and control, via the at least one throttle, flow rate of the solution from the extraction vessel to the separation vessel based at least partly on the processed real time information about the at least one of the plurality of compounds.

2. An extraction system according to claim 1 wherein the processed real time information comprises a concentration value of the at least one of the plurality of compounds in the solution.

3. An extraction system according to claim 1 or 2 wherein the processed real time information comprises a ratio of concentration values of at least two of the plurality of compounds in the solution.

4. An extraction system according to any one of claims 1 to 3 wherein the processed real time information comprises a rate change of ratio of concentration values of at least two of the plurality of compounds in the solution.

5. An extraction system according to any one of claims 1 to 4 wherein the real time information includes real time information about the at least one target compound.

6. An extraction system according to any one of claims 1 to 4 wherein the real time information excludes real time information about the at least one target compound.

7. An extraction system according to any one of claims 1 to 6 wherein the controller is configured to actuate the at least one throttle to stop flow of the solution from the extraction vessel to the separation means when the processed real time information indicates completion of extraction of the at least one target compound from the botanical matter.

8. An extraction system according to any one of claims 1 to 8 further comprising a heater and/or cooler in thermal communication with the extraction vessel, wherein the controller is in operative communication with the heater and/or cooler, wherein the controller is configured to actuate the heater and/or cooler to adjust the temperature of the extraction vessel in response to the processed real time information.

9. An extraction system according to any one of claims 1 to 8 further comprising a pump for pumping the solvent from the solvent source to the extraction vessel, and further comprising a valve between the solvent source and the extraction vessel for controlling flow of the solvent from the solvent source to the extraction vessel, wherein the controller is in operative communication with the pump and/or the valve, wherein the controller is configured to actuate the pump and/or the valve to adjust the pressure in the extraction vessel in response to the processed real time information.

10. An extraction system according to any one of claims 1 to 9 wherein the detector is configured to obtain real time information about the at least one of the plurality of compounds in the solution in an isolated section of the extraction vessel, the isolated section defined between a first throttle and a second throttle, wherein the controller is configured to actuate the first throttle and the second throttle to isolate a portion of the solution in the isolated section.

11. An extraction system according to claim 10 comprising a heater and/or cooler in thermal communication with the isolated section, wherein the controller is in operative communication with the heater and/or cooler, wherein the controller is configured to actuate the heater and/or cooler to adjust the temperature of the isolated section in response to the processed real time information.

12. An extraction system according to claim 11 wherein the cooler is configured to adjust the temperature in the isolated section to between 31° C. to −56° C. to facilitate phase separation of the extracted compounds in the isolated solution for improved signal detection by the detector.

13. An extraction system according to claim 10 wherein the controller is configured to actuate the first throttle and the second throttle to adjust the pressure of the isolated section of the extraction vessel in response to the processed real time information.

14. An extraction system according to any one of claims 10 to 13 wherein the isolated section is part of a conduit section of the extraction vessel, and wherein the second throttle is the at least one throttle located between the extraction vessel and the separation vessel.

15. An extraction system according to claim 14 wherein the controller is configured to actuate the first throttle and the second throttle to reduce pressure in the isolated section to between 75 atm to 5 atm to facilitate phase separation of the extracted compounds in the isolated solution for improved signal detection by the detector.

16. An extraction system according to any one of claims 1 to 15 wherein the detector is selected from the group consisting of FTIR, NIR, LC-MS, GC-MS, UV absorbance, and UV fluorescence.

17. An extraction system according to claim 16 wherein the detector comprises FTIR, and wherein an FTIR probe is disposed proximal to the botanical matter in the extraction vessel.

18. An extraction system according to claim 17 wherein distance between the FTIR probe and the botanical matter ranges from 1 nm to 50 cm.

19. An extraction system according to any one of claims 1 to 18 wherein the extraction vessel and/or the separation vessel comprises a fixed volume.

20. An extraction system according to claim 19 wherein the extraction vessel and/or separation vessel each comprise a capped steel container or a plurality of capped steel containers.

21. An extraction system according to any one of claims 1 to 20 wherein the extraction vessel is pressurized.

22. An extraction system according to claim 21 wherein flow of the solution from the extraction vessel to the separation vessel is pressure driven.

23. An extraction system according to claim 22 wherein pressure in the separation vessel is lower than pressure in the extraction vessel to facilitate phase separation of the solution in the separation vessel.

24. An extraction system according to any one of claims 1 to 23 wherein the at least one throttle, the first throttle and/or the second throttle are valves.

25. A method for extracting at least one target compound from botanical matter comprising:
(a) depositing botanical matter into an extraction vessel;
(b) pressurizing a flow of solvent into the extraction vessel;
(c) extracting compounds from the botanical matter into the solvent to form a solution;
(d) detecting and measuring concentrations of at least two compounds in the solution from step (c);
(e) deriving a rate of change of ratios of the concentrations of the at least two compounds;
(f) adjusting at least one process parameter based on at least the rate of change from step (e);
(g) allowing the solution to flow into a separation vessel once extraction of the at least one target compound is complete; and
(h) separating the at least one target compound from the solution.

26. A method according to claim 25 wherein steps (d) to (f) are performed in real time.

27. A method according to claim 25 or 26 wherein the adjusting of step (f) is based also on a predetermined relative order of extraction of the at least two compounds and the at least one target compound.

28. A method according to any one of claims 25 to 27 wherein the adjusting step (f) is based on a computer algorithm that relates the rate of change of ratios to the at least one process parameter.

29. A method according to claim 28 wherein the computer algorithm utilizes a database developed at least in part by computational machine learning.

30. A method according to any one of claims 25 to 29 wherein the at least one process parameter is selected from the group consisting of length of extraction time, extraction vessel temperature and extraction vessel pressure.

31. A method according to claim 30 where the at least one process parameter is the length of extraction time, wherein the length of extraction time is regulated by at least one valve between the extraction vessel and the separation vessel, wherein the at least one valve is controlled by a controller receiving the concentration values from step (e) and deriving the rate of change of step (f).

32. A method according to claim 31 wherein the length of extraction time is regulated by a plurality of valves between the extraction vessel and the separation vessel, wherein the plurality of valves is controlled by a controller receiving the concentration values from step (e) and deriving the rate of change of step (f).

33. A method according to claim 32 where the at least one process parameter is the extraction vessel temperature, wherein the extraction vessel temperature is regulated by a heater and/or cooler in thermal communication with the extraction vessel, wherein the heater and/or cooler is controlled by a controller receiving the concentration values from step (e) and deriving the rate of change of step (f).

34. A method according to claim 32 where the at least one process parameter is the extraction vessel pressure, wherein the extraction vessel pressure is regulated by a pump for pumping the solvent from the solvent source to the extraction vessel, and a valve between the solvent source and the extraction vessel, wherein the pump and the valve are controlled by a controller receiving the concentration values from step (f) and deriving the rate of change of step (g).

35. A method according to any one of claims 25 to 34 wherein, if an adequate signal cannot be obtained at step (d), then:
(d)(i) isolating a section of the extraction vessel; and
(d)(ii) phase separating extracted compounds from the solvent in the isolated section to facilitate detection by the detector.

36. A method according to claim 35 wherein step (d)(ii) comprises lowering the temperature and/or pressure in the isolated section.

37. A method according to any one of claims 25 to 36 wherein the at least two compounds includes the at least one target compound.

38. A method according to any one of claims 25 to 37 wherein the at least two compounds excludes the at least one target compound.

39. A method according to any one of claims 25 to 38 wherein the detecting and measuring of step (d) is performed by FTIR, NIR, LC-MS, GC-MS, UV absorbance and/or UV fluorescence.

40. A method according to any one of claims 25 to 39 wherein the botanical matter comprises cannabis.

41. A method according to claim 40 wherein the at least one target compound is a cannabinoid.

42. A method according to claim 41 wherein the cannabinoid is tetrahydrocannabinol.

43. A method according to claim 41 wherein the cannabinoid is cannabidiol.

44. A method according to any one of claims 25 to 43 wherein the solvent comprises fluidic carbon dioxide.

45. A method according to any one of claims 25 to 44 wherein the solvent in the extraction vessel maintains a pressure in the range of 1 atm to 700 atm, or 74 atm to 340 atm.

46. A method according to any one of claims 25 to 45 wherein the duration of step (c) ranges from 1 minute to 1440 minutes, or 5 minutes to 60 minutes.

47. A method according to any one of claims 25 to 46 wherein the separation vessel maintains a pressure in the range of 1 atm to 70 atm, or 20 atm to 60 atm.

48. A method according to any one of claims 25 to 47 wherein in step (h) the at least one target compound is phase separated from the solution.

49. A method according to any one of claims 25 to 48 wherein the at least one target compound is collected from a lower ⅓ of the separation vessel.

50. Systems comprising any new inventive feature, combination of features or sub-combination of features disclosed herein.

51. Method comprising any new inventive step, act, combination of steps and/or sub-combination of steps and/or acts disclosed herein.
